(19) United States
(12) Patent Application Publication
(10) Pub. No.: US 2020/0262777 A1
(43) Pub. Date: Aug. 20, 2020

(54) METHOD FOR PREPARING HYDROXYBENZOPHENONE POLYGLYCOL ETHER (METH)ACRYLATE

(71) Applicant: BASF SE, Ludwigshafen am Rhein (DE)

(72) Inventors: Rupert KONRADI, Ladenburg (DE); Eva-Maria REIS-WALTHER, Breuberg (DE); Andrea MISSKE, Speyer (DE); Friederike FLEISCHHAKER, Ludwigshafen (DE); Hermann BERGMANN, Osnabrueck (DE)

(73) Assignee: BASF SE, Ludwigshafen am Rhein (DE)

(21) Appl. No.: 16/061,496

(22) PCT Filed: Dec. 13, 2016
(86) PCT No.: PCT/EP2016/080714

§ 371 Date: Jun. 12, 2018

Related U.S. Application Data
(60) Provisional application No. 62/267,301, filed on Dec. 15, 2015.

(30) Foreign Application Priority Data
Dec. 15, 2015 (DE) 10 2015 225 264.7

(51) Int. Cl. C07C 45/64

(52) U.S. Cl. CPC C07C 45/64

(57) ABSTRACT

A method for preparing hydroxybenzophenone polyglycol ether (meth)acrylate comprising the steps of
(i) reaction of hydroxybenzophenone with ethylene carbonate to give hydroxybenzophenone monoglycol ether,
(ii) ethoxylation of hydroxybenzophenone monoglycol ether with ethylene oxide to give hydroxybenzophenone polyglycol ether,
(iii) transesterification of hydroxybenzophenone polyglycol ether with alkyl (meth)acrylate to give hydroxybenzophenone polyglycol ether (meth)acrylate.

The invention relates to a method for preparing hydroxybenzophenone polyglycol ether (meth)acrylate.

Hydroxybenzophenone polyglycol ether (meth)acrylate is used, for example, as UV-crosslinker in coating compositions. Hydroxybenzophenone polyglycol ether (meth)acrylate can be prepared by (meth)acrylation of hydroxybenzophenone.

By introducing a polyethylene glycol chain between a benzophenone unit and a (meth)acrylate group, water-soluble products can be obtained. However, in the direct alkoxylation of hydroxybenzophenone and subsequent (meth)acrylation of the hydroxybenzophenone polyglycol ether formed, firstly dimers and trimers of hydroxybenzophenone are formed, polyethylene glycol bisbenzophenone for example, which cannot be further (meth)acrylated and in the later application in coating compositions can migrate out of the coating matrix. Secondly, polyethylene glycol is formed which can form a di(meth)acrylate during the methacrylation. This already acts as crosslinker without UV activation and leads to gel-like, water-insoluble products.

It is known from WO 00/15629 that hydroxybenzophenone reacts with activated polyethylene glycol to give hydroxybenzophenone polyglycol ether. Alkoxylated toluenesulfonic acid (polyethylene glycol monotosylate) is used as activated polyethylene glycol. This type of preparation, however, is very complex.

The object of the invention is to provide a simple to perform method for preparing hydroxybenzophenone polyglycol ether (meth)acrylate, which leads to a product of high purity.

The object is achieved by a method for preparing hydroxybenzophenone polyglycol ether (meth)acrylate comprising the steps of:
(i) reaction of hydroxybenzophenone with ethylene carbonate to give hydroxybenzophenone monoglycol ether;
(ii) ethoxylation of hydroxybenzophenone monoglycol ether with ethylene oxide to give hydroxybenzophenone polyglycol ether;
(iii) transesterification of hydroxybenzophenone polyglycol ether with alkyl (meth)acrylate to give hydroxybenzophenone polyglycol ether (meth)acrylate.

In the direct ethoxylation of hydroxybenzophenone with ethylene oxide, an aromatic alkoxide is generated in the first step using a base—for example KOH—which reacts with the ethylene oxide. The newly formed aliphatic alkoxide then reacts further. As a side reaction, the aromatic alkoxide can attack an ethoxylate of hydroxybenzophenone already formed and cleave the polyethylene glycol chain. Polyethylene glycol bisbenzophenone and polyethylene glycol are formed as product.

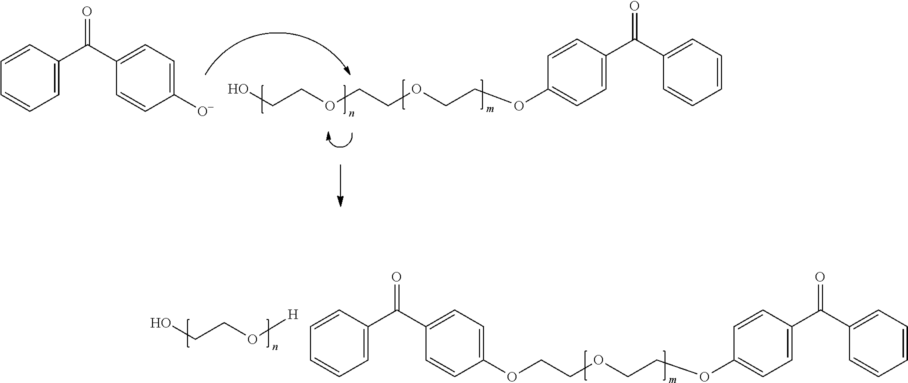

By tautomerization, an ethoxylation of the enolate can also take place as a further side reaction, which leads to trimers and higher homologs through subsequent reactions. By means of this reaction, the photocrosslinkable unit is lost.

The products were detected by mass spectrometry (coupled LC-MS).

It was found, surprisingly, that by introducing an ethylene oxide group upstream of the ethoxylation with ethylene oxide by reaction with ethylene carbonate, the formation of hydroxybenzophenone dimers and trimers and of polyethylene glycol is avoided.

Hydroxybenzophenone is generally 4-hydroxybenzophenone and 2-hydroxybenzophenone, preferably 4-hydroxybenzophenone.

In a first step (i), hydroxybenzophenone is reacted with ethylene carbonate to give hydroxybenzophenone monoglycol ether.

The reaction is generally carried out in the presence of sodium iodide as catalyst as described, for example, in WO2011/089385.

The reaction may be carried out in the presence of a separate solvent, toluene for example. The reaction may also be carried out without a separate solvent. In general, the reaction is carried out at a temperature in the range from 100 to 200° C., preferably 120 to 180° C. Ethylene carbonate is preferably used here in slight stoichiometric excess. After the end of the reaction, water is added to the monoethoxylate formed and the hydroxybenzophenone monoglycol ether is extracted with an organic solvent, for example toluene.

In a second step (ii), hydroxybenzophenone monoglycol ether is ethoxylated with ethylene oxide to give hydroxybenzophenone polyglycol ether. In general, 2 to 100, preferably 2 to 50, particularly preferably 4 to 50 mol of ethylene oxide are reacted per mole of hydroxybenzophenone monoglycol ether.

The ethoxylation may be carried out using gaseous ethylene oxide in the presence of basic catalysts at a pressure generally of 1 to 10 bar and temperatures of 80 to 200° C. Suitable basic catalysts are, for example, NaOH, KOH, sodium or potassium methoxide or potassium tert-butoxide. Preference is given to potassium tert-butoxide.

In a third step (iii), hydroxybenzophenone polyglycol ether is transesterified with alkyl (meth)acrylate to give hydroxybenzophenone polyglycol ether (meth)acrylate.

In general, the transesterification comprises the steps (iii-a) to (iii-d):
(iii-a) reacting alkyl (meth)acrylate with hydroxybenzophenone polyglycol ether in the presence of a catalyst and a stabilizer and in the presence of an azeotroping agent which forms an azeotrope with the alcohol bound in the alkyl (meth)acrylate,
(iii-b) continuously distilling off the azeotrope of azeotroping agent and alcohol, wherein steps (ii) and (iii) are conducted simultaneously until the hydroxybenzophenone polyglycol ether has been essentially fully converted,
(iii-c) removing the catalyst from the product mixture comprising hydroxybenzophenone polyglycol ether (meth)acrylate,
(iii-d) distilling unconverted alkyl (meth)acrylate and azeotroping agent out of the product mixture.

In step (iii-a), alkyl (meth)acrylate is reacted with hydroxybenzophenone polyglycol ether in the presence of a catalyst and a stabilizer and in the presence of an azeotroping agent which forms an azeotrope with the alcohol bound in the alkyl (meth)acrylate, wherein at the same time the azeotrope of azeotroping agent and alcohol is distilled off in step (iii-b) until hydroxybenzophenone polyglycol ether has been essentially fully converted. The transesterification thus consists of steps (iii-a) and (iii-b).

Suitable alkyl (meth)acrylates are the C₁-C₄-alkyl (meth)acrylates. In general, methyl (meth)acrylate or ethyl (meth)acrylate is used, with release of methanol or ethanol as alcohols in the transesterification reaction.

The reaction of alkyl (meth)acrylate with hydroxybenzophenone polyglycol ether is carried out in the presence of a homogeneous or heterogeneous catalyst. Useful are all Lewis acids or bases suitable for transesterification, for example, catalysts comprising tin, titanium or zirconium and inorganic salts.

Useful catalysts comprising tin are compounds comprising Sn(IV), for example dialkyltin dichloride, dialkyltin oxide, dialkyltin diacetate, bis(trialkyltin) oxide, bis(dibutylchlorotin) oxide, in particular dibutyltin dichloride, dimethyltin dichloride and dibutyltin oxide. The chloride-containing catalysts may be employed together with alkoxides, for example with sodium methoxide. Suitable catalysts comprising titanium(IV) or zirconium(IV) (here encompassed as metal(IV)) are metal(IV) tetraalkoxylates of linear or branched C₁-C₆ alcohols, preferably metal(IV) tetraisopropoxide, metal(IV) tetrabutoxide and also the metalates of the reactant alcohols used or mixtures thereof. Metalates substituted by different alcohols or by acetylacetonate are also possible.

The inorganic salt preferably has at least one anion selected from the group consisting of carbonate (CO₃²⁻), oxide (O²⁻, hydroxide (OH⁻), hydrogen carbonate (HCO₃⁻), phosphate (PO₄³⁻), hydrogen phosphate (HPO₄²⁻), dihydrogen phosphate (H₂PO₄⁻), nitrate (NO₃⁻), sulfate (SO₄²⁻), sulfite (SO₃²⁻) and carboxylate (R⁶COO⁻), where R⁶ is C₁-C₄-alkyl or C₆-aryl. Preference is given to oxide, hydroxide and phosphate or mixtures thereof, particular preference being given to phosphate. The inorganic salt preferably has at least one cation selected from the group consisting of alkali metals, alkaline earth metals, ammonium, cerium, iron, manganese, chromium, molybdenum, cobalt, nickel or zinc. Preference is given to alkali metals and alkaline earth metals and particular preference to lithium, sodium, potassium or calcium. Particularly preferred inorganic salts including hydrates thereof are LiOH, LiNO₃, Li₃PO₄, Na₃PO₄, K₃PO₄, Na₂CO₃, K₂CO₃ and CaO, especially preferred is K₃PO₄.

Particularly suitable are heterogeneous catalysts or homogeneous catalysts which can be converted into heterogeneous residues, as used in the transesterification methods described in, for example, DE 2 317 226 A1, DE 10 2004 036 930 A1 and WO2009/080380. The catalysts or residues of the catalysts are generally removed by filtration, electrofiltration, absorption, centrifugation or decantation.

The reaction of alkyl (meth)acrylate with hydroxybenzophenone polyglycol ether is additionally effected in the presence of one or more stabilizers (polymerization inhibitors). Preference is given to hydroquinone, hydroquinone monomethyl ether, phenothiazine, 4-hydroxy-2,2,6,6-tetramethylpiperidine N-oxyl, 4-oxo-2,2,6,6-tetramethylpiperidine N-oxyl, 2-tert-butylphenol, 4-tert-butylphenol, 2,4-di-tert-butylphenol, 2-tert-butyl-4-methylphenol, 6-tert-butyl-2,4-dimethylphenol, 2,6-di-tert-butyl-4-methylphenol and 2-methyl-4-tert-butylphenol. Particular preference is given to hydroquinone monomethyl ether (MeHQ).

Advantageously, oxygen may additionally be used as a polymerization inhibitor.

For further stabilization, an oxygenous gas, preferably air or a mixture of air and nitrogen (lean air), may be present.

The transesterification reaction (steps (iii-a) and (iii-b)) is generally conducted at a temperature of 60 to 140° C., preferably 70 to 110° C. In the course of this, an azeotrope of azeotroping agent and alcohol is distilled off continuously.

Suitable azeotroping agents which form an azeotropically boiling mixture with methanol or ethanol are, first of all, methyl acrylate and methyl methacrylate and also ethyl acrylate and ethyl methacrylate themselves. Suitable separate azeotroping agents include cyclohexane, methylcyclohexane, benzene, toluene, hexanes and heptanes, and mixtures thereof. Preference is given to methyl acrylate, methyl methacrylate, ethyl acrylate and ethyl methacrylate, and to mixtures of these with n-heptane and cyclohexane. The term azeotroping agent in this context encompasses the reactant itself and any separate solvent additionally used.

In a preferred embodiment, no separate solvent is used as azeotroping agent. In this case, the alkyl (meth)acrylate reactant itself serves as azeotroping agent.

The azeotroping agent may subsequently be replenished in the reactor. For this purpose, the azeotropic mixture of alcohol and azeotroping agent, in a preferred embodiment, is distilled off by means of a suitable column, stirred with water in a mixing vessel and then transferred into a phase separator, wherein the alcohol, generally methanol or ethanol, dissolves in water and the organic phase separates out as the upper layer. The organic phase is preferably returned to the reaction mixture via the top of the column and hence recirculated save for small losses. It is alternatively also possible to add fresh azeotroping agent and work up the azeotroping agent/alcohol mixture in a separate step or to wholly or partly dispense with replenishment of the azeotroping agent.

In general, alkyl (meth)acrylate is used in a stoichiometric excess. Preferably, the excess of methyl (meth)acrylate per hydroxyl group to be esterified is 5 to 2500 mol %, more preferably 300 to 2000 mol %.

The catalyst is used at a concentration of 0.1 to 10 mol %, based on the amount of hydroxybenzophenone polyglycol ether used, preferably at a concentration of 0.1 to 5 mol %.

The transesterification may be conducted at atmospheric pressure, but also under elevated pressure or reduced pressure. It is generally conducted at 300 to 1000 mbar, preferably at 300 to 700 mbar (atmospheric pressure=1000 mbar). The reaction time is generally 1 to 24 hours, preferably 3 to 18 hours and more preferably 3 to 10 hours. The transesterification (steps (iii-a) and (iii-b)) can be effected continuously, for example in a stirred tank cascade, or batchwise.

The reaction may be conducted in all reactors suitable for a reaction of this type. Such reactors are known to those skilled in the art. The reaction is preferably effected in a stirred tank reactor.

The batch can be mixed using any desired apparatuses, for example stirring apparatuses. The mixing can also be effected by feeding in a gas, preferably an oxygen-containing gas.

The alcohol formed, generally methanol or ethanol, is removed continuously or stepwise in a manner known per se by azeotropic distillation in the presence of an azeotroping agent. In addition, methanol may also be removed by stripping with a gas.

In a preferred embodiment, alcohol is removed from the azeotrope of azeotroping agent and alcohol distilled off in step (iii-b) by washing with water and the azeotroping agent is recycled into the reaction vessel.

Steps (iii-a) and (iii-b) are conducted until the hydroxybenzophenone polyglycol ether used has been essentially fully converted. This is the case when hydroxybenzophenone polyglycol ether has been converted to an extent of 95%, preferably to an extent of 97% and more preferably to an extent of 98%. The degree of conversion can be analyzed most simply via the determination of the OH number. This indicates the content of OH groups as a cumulative parameter in the unit mg KOH/g of substance and can be converted to percent by weight assuming a particular molar mass of the alcohol.

In step (iii-c), the catalyst is separated from the product mixture comprising hydroxybenzophenone polyglycol ether (meth)acrylate, for example by filtration or centrifugation.

The filtration can be conducted, for example, with a pressure suction filter. In process engineering terms, for a filtration in the method according to the invention, it is possible to use any filtration methods and apparatuses known per se, for example those described in Ullmann's Encyclopedia of Industrial Chemistry, 7th ed., 2013 Electronic Release, chapter: Filtration, 1. Fundamentals and Filtration 2. Equipment. For example, these may be cartridge filters, filter presses, pressure plate filters, bag filters or drum filters. Preference is given to using cartridge filters or pressure plate filters. The filtration can be conducted with or without filtering aids. Suitable filtering aids are filtering aids based on kieselguhr, perlite and cellulose.

Suitable centrifuges and also separators are known to the expert. In process engineering terms, for a centrifugation in the method according to the invention, it is possible to use any centrifugation methods and apparatuses known per se, for example those described in Ullmann's Encyclopedia of Industrial Chemistry, 7th ed., 2013 Electronic Release, chapter: Centrifuges, Filtering and Centrifuges, Sedimenting.

The removal of the catalyst can also be effected as an aqueous extraction by addition of water. For this purpose, the product mixture comprising as yet unconverted alkyl (meth)acrylate and any separate azeotroping agent, and also the stabilizer and the catalyst, is contacted with water. It is also possible to conduct two or more washing steps, for example three washing steps. The amount of washing water per washing step is generally 0.1 to 2 times and preferably 0.2 to 0.5 times the amount of product mixture.

The wash can be conducted, for example, in a stirred vessel or in another conventional apparatus, for example in a column or mixer-settler apparatus.

In process engineering terms, for a wash in the method according to the invention, it is possible to use any extraction and washing methods and apparatuses known per se, for example those described in Ullmann's Encyclopedia of Industrial Chemistry, 6th ed., 1999 Electronic Release, chapter “Liquid—Liquid Extraction—Apparatus”. For example, these may be single-stage or multistage, preferably single-stage, extractions and also extractions in cocurrent or countercurrent mode.

The washed reaction mixture is optionally admixed with a storage stabilizer, such that the desired concentration of stabilizer, for example 100 ppm, is attained in the target product. This concentration, which is adjustable as desired by this method, depends on the particular specification of the end product and for commercial alkyl (meth)acrylates, for example, is in the range from 15 to 200 ppm. The storage stabilizers used are generally stabilizers selected from the group of the phenols, for example 2,6-di-tert-butyl-4-methylphenol, 6-tert-butyl-2,4-dimethylphenol, hydroquinone and hydroquinone monomethyl ether, preferably hydroquinone monomethyl ether.

Subsequently, in a distillation step (iii-d), unconverted alkyl (meth)acrylate and any separate azeotroping agent, and also any water, are distilled out of the product mixture. This distillation is generally effected at a temperature of 40 to 100° C., preferably 60 to 80° C., and a variable pressure of 2 to 700 mbar. In addition, these components may also be removed by stripping with a gas, preferably an oxygenous gas.

The distillative removal is effected, for example, in a stirred tank with jacket heating and/or internal heating coils under reduced pressure.

It will be appreciated that the distillation can also be effected in a falling-film or thin-film evaporator. To this end, the reaction mixture is passed through the apparatus, preferably repeatedly in circulation, under reduced pressure, for example at 20 to 700 mbar, preferably 30 to 500 mbar and more preferably from 50 to 150 mbar, and a temperature of 40 to 80° C.

An inert gas, preferably an oxygenous gas and more preferably air or a mixture of air and nitrogen (lean air), may advantageously be introduced into the distillation apparatus, for example 0.1 to 1, preferably 0.2 to 0.8 and more preferably 0.3 to 0.7 m³/m³h, based on the volume of the reaction mixture.

Once steps (iii-c), and (iii-d) have been carried out, there remains a product in the form of a bottoms product which is obtained at high purity. This is generally at least 95% by weight, preferably at least 98% by weight.

The invention also provides hydroxybenzophenone polyglycol ether (meth)acrylates having 5 to 100 ethylene oxide units and also use thereof as UV crosslinkers in radiation-curable coating compositions.

The invention is illustrated by the following examples.

### EXAMPLES

### Example 1 Synthesis of Hydroxyethoxybenzophenone (HEBP)

94.2 g (1.07 mol) of ethylene carbonate, 206 g (1.02 mol) of 4-hydroxybenzophenone (HBP), 4.5 g (0.030 mol) of sodium iodide and 8.0 g of toluene are initially charged and heated slowly with stirring to 165° C. Evolution of gas starts from 135° C. The mixture is stirred at 165° C. for 1 h. After cooling to room temperature, water and toluene are added, the phases separated and the organic phase is washed with water, dried and concentrated. The solid is taken up in cold toluene and filtered off with suction. This gives 194 g (0.802 mol, 79% of theory) of a light beige solid.

### Example 2 Synthesis of HEBP+4 EO

242 g (1.00 mol) of HEBP with 834 mg (7.43 mmol) of KOtBu are initially charged in an autoclave at 80° C. 176 g (4.00 mol) of ethylene oxide are metered in at 120° C. and the mixture is stirred at this temperature for 10 h. After cooling to room temperature, the mixture is freed of volatile constituents on a rotary evaporator. This gives 415 g of a brown liquid.

OHN=139.6 mg KOH/g (theory: 134.4 mg KOH/g)

The OH number deviates from theory by 4%.

### Example 3 Synthesis of HEBP+9 EO

80.7 g (0.33 mol) of HEBP and 425 mg (3.79 mmol) of KOtBu are initially charged in an autoclave at 80° C. 132 g (3.00 mol) of ethylene oxide are metered in at 120° C. and the mixture is stirred at this temperature for 10 h. After cooling to room temperature, the mixture is freed of volatile constituents on a rotary evaporator. This gives 210 g of a brown liquid.

OHN=84.3 mg KOH/g (theory: 87.8 mg KOH/g)

The OH number deviates from theory by 4%.

### Example 4 Synthesis of HEBP+10 EO

80.7 g (0.33 mol) of HEBP with 720 mg (6.43 mmol) of KOtBu are initially charged in an autoclave at 80° C. 279 g (6.33 mol) of ethylene oxide are metered in at 120° C. and the mixture is stirred at this temperature for 3 h. After cooling to room temperature, the mixture is freed of volatile constituents on a rotary evaporator. This gives 368 g of a brown solid.

OHN=53.1 mg KOH/g (theory: 52.0 mg KOH/g)

The OH number deviates from theory by 2%.

### Example 5 Synthesis of HBP+5 EO Methacrylate

The transesterification is effected with introduction of air in a 750 mL jacketed reactor equipped with an anchor stirrer, an air inlet, a separating column and a liquid divider. This apparatus is initially charged with 347 g of HBP*5EO, 0.1 g of methylhydroquinone (MEHQ) and 600 g of methyl methacrylate (MMA, stabilized with 15 ppm of MEHQ) at room temperature. 9.1 g of potassium phosphate are added and the reaction mixture is heated at a bath temperature of 100° C. A pressure of 300 mbar (abs.) is established and an azeotrope of methanol and MMA is distilled off continuously, in the course of which the bottoms temperature stabilizes at 63° C. to 68° C. The reflux ratio is variable from 2:1 to 10:1 (reflux:output). After the reaction has ended, the product is filtered through a paper filter and the reaction mixture is concentrated under reduced pressure. The conversion is determined via TAI NMR as >99%. The OH number is <2. The stabilizer content is 170 ppm MEHQ (determined by HPLC).

### Example 6 Synthesis of HBP*10 EO Methacrylate

The apparatus of Example 6 is initially charged with 163 g of benzophenone*10EO, 0.06 g of methylhydroquinone (MEHQ) and 400 g of methyl methacrylate (MMA, stabilized with 15 ppm of MEHQ) at room temperature. 2.9 g of potassium phosphate are added and the reaction mixture is heated at a bath temperature of 100° C. A pressure of 300 mbar (abs.) is established and an azeotrope of methanol and MMA is distilled off continuously, in the course of which the bottoms temperature stabilizes at 66° C. The reflux ratio is variable from 2:1 to 25:1 (reflux:output). After the reaction has ended, the product is filtered through a paper filter and the reaction mixture is concentrated under reduced pressure. The conversion is >99%, determined via TAI NMR. The

OH number is <2. The stabilizer content is 260 ppm MEHQ (determined by HPLC).

### Example 7 Synthesis of HBP*20 EO Methacrylate

The apparatus of Example 6 is initially charged with 300 g of benzophenone*20EO, 0.08 g of methylhydroquinone (MEHQ) and 600 g of methyl methacrylate (MMA, stabilized with 15 ppm of MEHQ) at room temperature. 4.8 g of potassium phosphate are added and the reaction mixture is heated at a bath temperature of 100° C. A pressure of 300 mbar (abs.) is established and an azeotrope of methanol and MMA is distilled off continuously, in the course of which the bottoms temperature stabilizes at 66° C. The reflux ratio is variable from 5:1 to 10:1 (reflux:output). After the reaction has ended, the product is filtered through a paper filter and the reaction mixture is concentrated under reduced pressure. The conversion is >99%, determined via TAI NMR.

### Comparative Example 1 Direct Ethoxylation of Hydroxybenzophenone (HBP) with 10 EO

496 g (2.50 mol) of hydroxybenzophenone are initially charged in an autoclave with 6.38 g (56.9 mmol KOH) of an aqueous KOH solution and the mixture dewatered at 140° C. and 25 mbar for 2 h. 1100 g (25.0 mol) of ethylene oxide are metered in at 140° C. over 12 h and the mixture is stirred at this temperature for 8 h. After cooling to room temperature, the mixture is freed of volatile constituents on a rotary evaporator. This gives 1590 g of a brown liquid.

OHN=105.8 mg KOH/g (theory: 87.8 mg KOH/g)

The OH number deviates from theory by 21%.

The substance was analyzed by means of coupled LC-MS. In addition to the desired product, the mixture comprises polyethylene glycol, polyethylene glycol bisbenzophenone and also trimers and higher homologs of hydroxybenzophenone.

### Comparative Example 2 Direct Ethoxylation of HBP with 20 EO

297 g (1.50 mol) of hydroxybenzophenone are initially charged in an autoclave with 6.46 g (57.6 mmol KOH) of an aqueous KOH solution and the mixture dewatered at 140° C. and 25 mbar for 2 h. 1320 g (30.0 mol) of ethylene oxide are metered in at 140° C. over 12 h and the mixture is stirred at this temperature for 8 h. After cooling to room temperature, the mixture is freed of volatile constituents on a rotary evaporator. This gives 1610 g of a brown solid.

OHN=79.0 mg KOH/g (theory: 52.0 mg KOH/g)

The OH number deviates from theory by 52%.

The substance was analyzed by means of coupled LC-MS. In addition to the desired product, the mixture comprises polyethylene glycol, polyethylene glycol bisbenzophenone and also trimers and higher homologs of hydroxybenzophenone.

Comparison of OH Numbers

| Experiment | 2 | 3 | 4 | C1 | C2 |
|---|---|---|---|---|---|
| OHN found | 139.6 | 84.3 | 53.1 | 105.8 | 79 |
| OHN theory | 134.4 | 87.8 | 52 | 87.8 | 52 |
| Deviation | 4 | −4 | 2 | 21 | 52 |

1: A method for preparing hydroxybenzophenone polyglycol ether (meth)acrylate, the method comprising
(i) reacting hydroxybenzophenone with ethylene carbonate to give hydroxybenzophenone monoglycol ether,
(ii) performing an ethoxylation of hydroxybenzophenone monoglycol ether with ethylene oxide to give hydroxybenzophenone polyglycol ether, and
(iii) performing a transesterification of hydroxybenzophenone polyglycol ether with an alkyl (meth)acrylate to give hydroxybenzophenone polyglycol ether (meth)acrylate.

2: The method according to claim 1, wherein the reacting (i) is carried out in the presence of sodium iodide.

3: The method according to claim 1, wherein 2 to 50 mol of ethylene oxide per mole of hydroxybenzophenone monoglycol ether are reacted in the ethoxylation.

4: The method according to claim 1, wherein the ethoxylation is carried out in the presence of potassium tert-butoxide as catalyst.

5: The method according to claim 1, wherein the transesterification comprises:
(iii-a) reacting the alkyl (meth)acrylate with hydroxybenzophenone polyglycol ether in the presence of a catalyst and a stabilizer and in the presence of an azeotroping agent which forms an azeotrope with the alcohol bound in the alkyl (meth)acrylate,
(iii-b) continuously distilling off the azeotrope of the azeotroping agent and alcohol, wherein the ethoxylation and the transesterification are conducted simultaneously until the hydroxybenzophenone polyglycol ether has been essentially fully converted,
(iii-c) removing the catalyst from a product mixture comprising hydroxybenzophenone polyglycol ether (meth)acrylate, and
(iii-d) distilling unconverted alkyl (meth)acrylate and azeotroping agent out of the product mixture.

6: The method according to claim 5, wherein the azeotroping agent is the alkyl (meth)acrylate.

7: The method according to claim 5, wherein the azeotroping agent is a separate solvent other than an alkyl (meth)acrylate.

8: The method according to claim 7, wherein the azeotroping agent is selected from the group consisting of n-heptane and cyclohexane.

9: The method according to claim 5, wherein the catalyst used in (iii-a) is potassium phosphate.

10: The method according to claim 5, wherein the stabilizer used in (iii-a) is hydroquinone monomethyl ether.

11: The method according to claim 1, wherein the alkyl (meth)acrylate used in the transesterification is methyl or ethyl (meth)acrylate.

12: A hydroxybenzophenone polyglycol ether (meth)acrylate, having 5 to 100 ethylene oxide units.

13: A hydroxybenzophenone polyglycol ether (meth)acrylate, prepared by the method according to claim 1.

14. (canceled)
